(19) United States
(12) Patent Application Publication
(10) Pub. No.: US 2024/0055080 A1
(43) Pub. Date: Feb. 15, 2024

(54) PHASE STABILITY OF POLYMER MATERIAL

(71) Applicants: BASF SE, Ludwigshafen Am Rhein (DE); BASF ADVANCED CHEMICALS CO., LTD., Shanghai (CN)

(72) Inventors: Zhe Xiong Yang, Shanghai (CN); Huan Huan Gao, Shanghai (CN); Hai Long Lv, Shanghai (CN); Ke Wang, Shanghai (CN); Ye Fan Wu, Shanghai (CN); Ke Shi, Shanghai (CN); Johannes Barth, Shanghai (CN); Andreas Kuehner, Shanghai (CN); Xuyuan Peng-Poehler, Ludwigshafen (DE); Yi Zhou Zheng, Shanghai (CN)

(21) Appl. No.: 18/267,603

(22) PCT Filed: Dec. 17, 2021
(86) PCT No.: PCT/EP2021/086587

§ 371 Date: Jun. 15, 2023

(30) Foreign Application Priority Data
Dec. 18, 2020 (WO) PCT/CN2020/137559

(51) Int. Cl. G16C 20/30; G16C 20/70; G16C 20/10

(52) U.S. Cl. CPC G16C 20/30; G16C 20/70; G16C 20/10

(57) ABSTRACT

A computer implemented method of predicting a phase stability parameter for a polymer material comprising the steps of providing to a computer processor via a communication interface a digital representation of the polymer material; providing to the processor via the communication interface a data driven model parametrized on a digital representation of historical polymer material, and historical phase stability parameters; determining with the computer processor a phase stability parameter for the polymer material based on the provided data driven model, and the digital representation of the polymer material; providing via the communication interface the determined phase stability parameter.

### FIELD

The invention relates to a computer implemented method for determining phase stability of polymer materials, a system for predicting phase stability of polymer materials and a computer program product for determining phase stability of polymer materials. The invention also relates to use of the determined phase stability of polymer materials for storage advice, for prescreening of polymer material formulations. The invention further relates to a method, a system and a computer program product for optimizing a polymer material based on target requirements. The invention further relates to a system comprising a polymer material and a stability parameter derived by the computer implemented method for determining phase stability of polymer materials, a system for determining phase stability of polymer materials and a computer program product for determining phase stability of polymer materials.

### BACKGROUND

Polymers are widely used along the value chain in chemical industries. One aspect in polymer chemistry is stability.

In particular, this stability plays a role in polymer blends which are often used as raw material in industrial production of polymers such as foams, coatings, etc.; in polymer melts which for example are frequently used in manufacturing of filter materials, adsorbents, etc.; and in solid phase polymers, solid phase polymers are mostly present in end products.

In industrial production polymer blends are frequently shipped from the initial manufacturer to the customer and stored at various places in between, leading to a time lag between production of the polymer blend and use of the polymer blend. Usability of the polymer blend depends on stability of the blend, which is often closely tied to the phase stability of the polymer blend. A problem that may occur with polymer blends is phase separation which is discussed in various textbooks (e.g. Wenbing Hu “Polymer physics—a molecular approach”, DOI 10.1007/978-3-7091-0670-9, Springer 2013). Currently the phase stability of polymer blends is experimentally tested by storing the polymer blends under defined conditions and probing the phase stability over time. This is very time consuming and costly, in particular for newly formulated polymer blends.

For polymer melts the situation is slightly different, polymer melts are often used on continuous processes, instabilities usually require shut down of the process. The stability of polymer melts may be determined by producing a polymer melt and determine how long this melt is stable.

For solid phase polymers the stability determines the lifetime of the product. Instabilities may influence the performance of the polymer product. The stability of solid phase polymers may be determined by the phase stability of the solid phase polymer. Currently the phase stability of solid phase polymers is experimentally tested by storing the solid phase polymer under defined conditions and probing the stability over time by assessing whether the properties are still maintained.

### SUMMARY

To address the above-mentioned problems in a perspective the following is proposed:
A computer implemented method of determining a phase stability parameter for a polymer material comprising the steps of
   providing to a computer processor via a communication interface a digital representation of the polymer material associated with a synthesis specification;
   providing to the processor via the communication interface a data driven model parametrized on a digital representation of historical polymer material, and historical phase stability parameters; and a pairwise interaction parameter associated with the digital representation of the polymer material,
   determining with the computer processor a phase stability parameter for the polymer material based on the provided data driven model, and the digital representation of the polymer material
   providing via the communication interface the determined phase stability parameter.

The proposed method greatly reduces the time to assess a phase stability parameter, by reducing the necessity of performing experiments for each polymer material. In addition, the proposed method further reduces the burden to perform experiments that are otherwise required to assess the phase stability parameter. This increases reliability that the polymer material will be phase stable until use in production. This further allows a faster validation if a polymer material meets a target phase stability.

Further disclosed is:
A computing apparatus comprising:
a computer processor; a communication interface and
a memory storing instructions that, when executed by the processor, configure the apparatus to perform the steps of
   providing to a computer processor via a communication interface a digital representation of the polymer material associated with a synthesis specification;
   providing to the processor via the communication interface a data driven model parametrized on
      a digital representation of historical polymer material, and
      historical phase stability parameters;
   determining with the computer processor a phase stability parameter for the polymer material based on
      the provided data driven model, and
      the digital representation of the polymer material
   providing via the communication interface the determined phase stability parameter.

Further disclosed is:
A non-transitory computer-readable storage medium, the computer-readable storage medium including instructions that when executed by a computer, cause the computer to perform the steps of
   providing to a computer processor via a communication interface a digital representation of the polymer material associated with a synthesis specification;
   providing to the processor via the communication interface a data driven model parametrized on
      a digital representation of historical polymer material, and
      historical phase stability parameters;
   determining with the computer processor a phase stability parameter for the polymer material based on
      the provided data driven model, and
      the digital representation of the polymer material
   providing via the communication interface the determined phase stability parameter.
      Further the use of the method disclosed herein for screening of polymer material is disclosed. Further disclosed is a system comprising a polymer material and a shelf-life, wherein the shelf- life was validated depending on the phase stability parameter determined according the method disclosed herein.
      Use of the method disclosed herein for screening polymer material according to a stability criterion.
      Further disclosed is a system comprising a polymer material, and a phase stability parameter, wherein the phase stability parameter is determined according to the method disclosed herein. Further disclosed is a system comprising at least one polymer and/or prepolymer and a formulation comprising the at least one polymer and/or prepolymer, and a phase stability parameter, wherein the phase stability parameter is determined according to the method disclosed herein.
      Further disclosed is:
      A computer implemented method for determining a formulation of a polymer material, comprising the steps of:
providing to a computer processor via a communication interface an initial digital representation of a polymer material,
providing to a computer processor via a communication interface a target phase stability parameter;
providing to the processor via the communication interface a data driven model parametrized on a digital representation of historical liquids containing polymers and historical phase stability parameters;
determining with the computer processor a determined phase stability parameter for the polymer material based on the provided data driven model, and the digital representation of the polymer material;
optimizing the storage time, by modifying the ratio of the ingredients in the initial digital representation of the polymer material until the desired shelf life is reached;
providing a target formulation comprising the ratio of the ingredients determined in the step of optimizing.
   For the optimizing step, an objective function may be defined comprising the target phase stability parameter and the determined phase stability parameter. The objective function may then be minimized by changing the concentrations of the ingredients.
   Further disclosed is a formulation of a polymer material, wherein in the formulation was derived according the method of determining a formulation disclosed herein. Further disclosed is a client device for generating a request to initiate the determination of a phase stability parameter of a polymer material at a server device, wherein the client device is configured to provide a digital representation of the polymer material and the target storage time to a server device.
   Further disclosed is a method for validating the phase stability of a polymer material, comprising the steps of providing a target phase stability parameter, providing to a computer processor via a communication interface a digital representation of the polymer material associated with a synthesis specification; providing to the processor via the communication interface a data driven model parametrized on a digital representation of historical polymer material, and historical phase stability parameters; and a pairwise interaction parameter associated with the digital representation of the polymer material, determining with the computer processor a phase stability parameter for the polymer material based on the provided data driven model, and the digital representation of the polymer material providing via the communication interface the determined phase stability parameter, comparing the target phase stability parameter and the determined phase stability parameter, validating the polymer material if the determined phase stability parameter meets the target phase stability parameter within a boundary, providing a validation signal suitable for controlling synthesis of the polymer material.

The phase stability parameter may be a shelf life. A shelf life may be in particular interesting, when the polymer material is related to polymer blends. Polymer blends are often sold to customers with a guaranteed shelf life.

The phase stability parameter may be a stability lifetime, this may be a more important parameter for polymer melts. Polymer melts are rarely stored as melts for a longer time, therefore the stability lifetime may be shorter than the shelf life of polymer blends.

The phase stability parameter may be a life cycle time, this is in particular useful, when solid phase polymers are considered. Over time the solid phase polymers may change properties which than may determine the life cycle time.

The phase stability parameter may be related to a probability, a stability property, that the polymer material is stable after a time period, e.g. a time period t.

The phase stability parameter may be a classifier, “stable” “non-stable”, this may be derived from a threshold to the probability of stability.

In an aspect providing the digital representation of the polymer material may comprise providing a polymer material formulation, the polymer material formulation may comprise a polymer identifier for each of the polymers contained in the polymer material, and their respective concentration. In some examples the formulation may further comprise additives. A polymer identifier may e.g. be the chemical name, the structure formula, the CAS number, the SMILES (The simplified molecular-input line-entry system) string, the lab sample label, or a brand name. INCI name, kinetic model with monomer concentration and process conditions.

Polymer material formulations are easily interpretable, this enables use of the method by experimentalists rather than highly qualified specialists.

In an aspect the step of providing the digital representation comprises deriving sub-units from the formulation of the polymer material. The ingredients of the polymer material may be by reflected by sub-units and a concentration of respective sub-units may reflect the concentration of the ingredients and the concentration of each sub-unit in each ingredient. The concentration of the sub-units may be based on the amount of a specific sub-unit in a polymer and the concentration of that polymer in the polymer material. In an example, the sub-units for polymers may be stored in a database and retrieved via the communication database, in other examples the sub-units may be derived by decomposition of the polymer structure in predefined sub-units. The concentration of each sub-unit may be derived from the concentration of each sub-unit in the polymers and the concentration of polymers. Deriving the concentration may be a simple calculation. In some cases, the sub-units may be derived from a polymer synthesis recipe or from a NMR structural analysis result.

In an aspect, providing the digital representation may refer to reflecting the ingredients of the polymer material in sub-units and a concentration of respective sub-units reflects the concentration of the ingredients and the concentration of each sub-unit in each ingredient.

Polymer materials are very difficult to describe by molecular dynamic simulations. Polymer materials often have strongly varying formulations. Reflecting the ingredients of the polymer material in sub-units allows reducing the number of parameters needed for describing polymer materials. By reducing the parameters needed to describe the polymer material it is possible to reduce the training data set. Which is an important factor in machine learning.

The description of polymers in sub-units has further the advantage that the sub-units may be treated as soft matter particles. A sub-unit may consist of parts of the polymer providing similar properties. These properties may be chemical properties or physical properties, these properties may be functional groups. Similar properties may be properties describe indistinguishable parts of the polymer. Even two different polymers may have identical sub-units. One can think of the sub-units as LEGO blocks.

In an aspect the sub-units may consist of groups of atoms. In an aspect the sub-units may be formed by repeating units. Repeating units are a well-known concept in polymer chemistry and generally refer to groups of atoms that have similar properties. Using the concept of repeating units allows higher comparability with other methods used in polymer chemistry, like molecular modelling. This increases the accuracy of the determined phase stability. This increases reliability that the polymer material will be phase stable until use in production.

In an aspect, a parameter for pairwise interaction between sub-units is provided. Use of a pairwise interactions parameter reduces complexity and results in a more reliable and accurate determination of the phase stability. This increases reliability that the polymer material will be phase stable until use in production. This enables a faster validation if a polymer material meets a target phase stability.

[symbol] is a well-established concept for example from molecular dynamic simulations. It was found that this can surprisingly also be applied to determine the phase stability of polymers. An advantage is that the number of parameters is greatly reduced compared to a full molecular description of the polymer material. This allows for a very simple representation of the polymer material.

The pairwise interaction may be described by a single interaction parameter a_{i,j}. The interaction parameter a_{i,j} may be the a repulsion amplitude describing the interaction between sub units i and j.

In an aspect the digital representation of the polymer material may be based on pairwise interactions of sub-units of the polymers in the polymer material.

In an aspect the digital representation based on the pairwise interactions of sub-units may be generated by sampling with a computer processor two sub-units from the sub-units contained in the polymer material and determining the pairwise interaction between the sampled sub-units.

The sampling of two sub-units from the sub-units contained in the polymer material and determining the pairwise interaction between the sampled sub-units has the advantage that the probability of the concentration of the sub-units will be reflected due to the stochastic effect of sampling and a feature vector is easily derived even for complex polymer material.

In an aspect the step of generating the digital representation of the polymer may comprise repeating the step of sampling and determining N times, wherein N is >=1.

The step of determining with the computer processor a phase stability parameter for the digital representation of the polymer material based on the provided data driven model, the digital representation of the polymer material and the target storage time may result in the probability that the polymer material is phase separated after the target storage time. The phase stability parameter may be the probability. In an alternative a threshold for a probability may be defined and the phase stability parameter is then determined by a binary classification that represents stable or not stable after the target storage time.

In an aspect the digital representation of the polymer material based on pairwise interactions of sub-units of the polymers in the polymer material may be generated by sampling with a computer processor two sub-units from the sub-units describing the polymer material as described above.

In an aspect the step of sampling and determining is repeated various times, for example N times, wherein N is >=1. Repeated sampling better reflects the concentration of sub-units in the polymer material. A larger sampling provides a more realistic picture of the true concentration of the sub-units in the polymer material. In a very simplistic understanding. The polymer material can be understood as a reservoir of sub-units with a specific distribution of the sub-units depending on the polymer concentration and the polymers in the polymer material. Sampling may refer to randomly picking two sub-units from the reservoir of sub-units. Subsequently the pairwise interaction between the two sub-units are determined. In a second sampling step, again two sub-units are picked randomly and then their pairwise interaction is determined. The probability of picking a sub-unit from the reservoir depends on the concentration of the sub-unit in the reservoir, which is determined by the polymers in the polymer material and the concentration of the polymers in the polymer material. There is a trade of between accuracy and the number of sample steps. A suitable number of samples is in the range of 10 to 10000, depending on the complexity of the polymer material. The complex description of the polymer material is now simplified into a single vector of the length N, which greatly reduces the required training data set. In addition, vector calculations are easy.

In an aspect, the pairwise interactions may be sorted in and ascending order. In another example the pairwise interactions may be sorted in a descending order.

In an aspect pairwise interaction parameters between sub-units of polymers may be stored in a database. The stored pairwise interaction between sub-units may be previously be determined by experiments, ab initio calculations, etc., for further details it is referred to Multiscale Materials Modeling in an Industrial Environment, DOI: 10.1146/annurev-chembioeng-080615-033615.

In an aspect the pairwise interaction of the sample may be determined by retrieving via the communication interface the pairwise interaction for a pair of sub-units from the database. Storing the pairwise interaction for each pair of sub-units in a data base is efficient as the data is then readily available when needed.

In an aspect the step of providing via the communication interface a phase stability parameter may comprise providing a shelf lifetime.

In an aspect, the method may further comprise the step of providing a target storage time. In that case the data driven model is further parametrized on historical storage times. In this example, the phase stability parameter may refer to a probability that the polymer material is phase stable after the target storage time.

In an aspect a maximum storage time may be determined, by iteratively increasing the target storage time until the phase stability parameter meets a threshold.

In an aspect the data driven model may be trained on the digital representations of historical polymer material, wherein the digital representation of historical polymer materials comprises sub-units for each polymer in the historical polymer material. A sub-unit may consist of parts of the historical polymer providing similar properties. These properties may be chemical properties or physical properties. Similar properties may be properties describe indistinguishable parts of the polymer. The sub-units may consist of groups of atoms or functional units. The sub-units may be formed by repeating units. The data driven model may be trained using any method suitable for classification. In an example the data driven model may be trained using gradient boosting decision tree, logistic regression, random forest, etc.

In an aspect the historical polymer materials may be described as a pair wise interaction between the sub-units. By that the polymer materials may be described as a function of sub-unit concentrations and interactions. Suitable parameters for describing the interaction may be repulsion amplitudes. The digital representation of the historical polymer material may be similar digital representation provided via the communication interface. In an aspect, the pairwise interactions may be sorted in and ascending order. In another example the pairwise interactions may be sorted in a descending order.

According to an aspect, a computer program or a computer program product or computer readable non-volatile storage medium is disclosed comprising computer readable instructions, which when loaded and executed by a computer processor perform the methods disclosed herein.

Any disclosure and embodiments described herein relate to the methods, the systems, the treatment devices, the computer program element lined out above and vice versa. Advantageously, the benefits provided by any of the embodiments and examples equally apply to all other embodiments and examples and vice versa.
As used herein “determining” also includes “initiating or causing to determine”, “generating” also includes “initiating or causing to generate” and “provding” also includes “initiating or causing to determine, generate, select, send or receive”. “Initiating or causing to perform an action” includes any processing signal that triggers a computing device to perform the respective action.

### BRIEF SUMMARY

### Brief Description of the Several Views of the Drawings

To easily identify the discussion of any particular element or act, the most significant digit or digits in a reference number refer to the figure number in which that element is first introduced.

FIG. 1 illustrates a routine in accordance with one embodiment.

FIG. 2 a) illustrates a polymer consisting of sub-units FIG. 2 b illustrates the polymer sub-units

FIG. 3 illustrates an aspect of the subject matter in accordance with one embodiment.

FIG. 4 illustrates client server setup for the proposed methods

### DETAILED DESCRIPTION

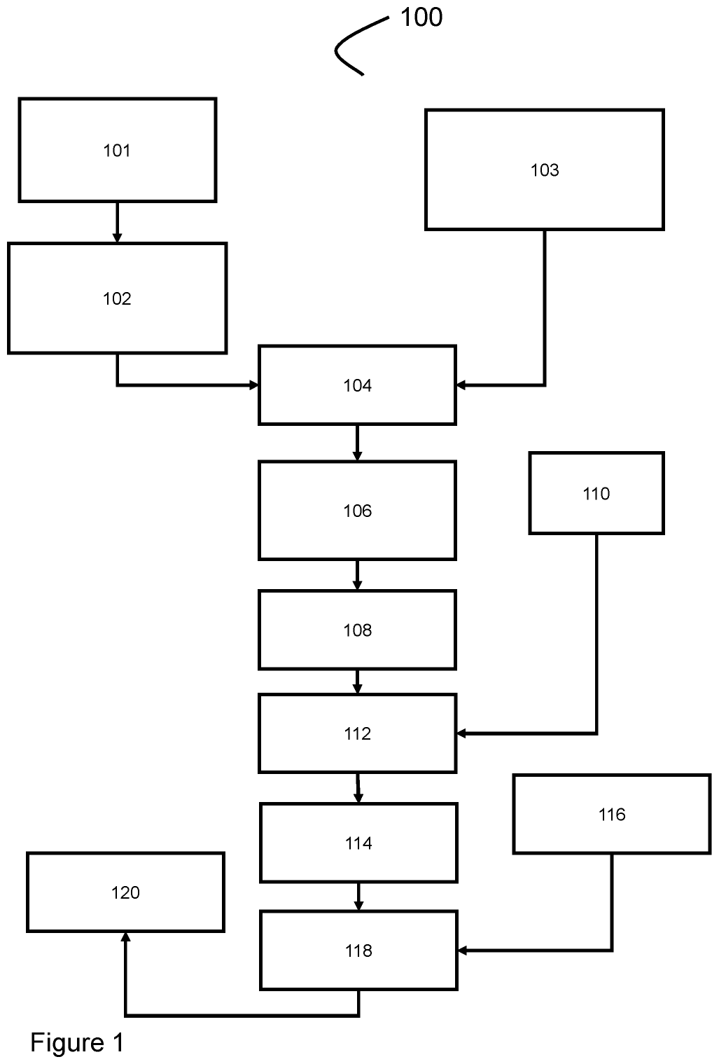

FIG. 1 depicts a non-liming embodiment of the computer implemented method of determining a phase stability parameter of a polymer material in this example, the polymer material may be a polymer blend. In other examples, the polymer material may be a polymer melt or a solid phase polymer. In block 102, routine 100 provides to a computer processor via a communication interface a digital representation of the polymer blend in this example the digital representation is a formulation of the polymer blend. In this example, a target storage time is further provided to the computer processor via the communication interface, this step is generally optional. In this example providing the digital representation comprises two polymers represented by their respective CAS number and the concentration of each of the polymers in the polymer blend. In accordance with the invention other digital representations may be used. Step 102 may be initated by a request step 101.

In optional block 104 sub-units are derived from the formulation of the polymer blend reflecting the ingredients of the polymer material in sub-units and reflecting the concentration of the ingredients by the concentration of the respective sub-units.

In this example this is performed by retrieving in block 103 sub-units for each of the ingredients in the polymer blend from a data base, calculate the concentration of the sub-units based on the retrieved sub-units and the concentration of the ingredients in the polymer material.

In Block 106 the digital representation is based on a parameter of pairwise interaction of sub-units of the ingredients of the polymer material, in this example the polymers in the polymer blend. Block 106 may further comprises the step of sampling two sub-units from the sub-units contained in the polymer blend and determining the pairwise interaction between the sampled sub-units.

In block 108, routine 100 repeats the step of sampling N times. In block 110, routine 100 retrieves the pairwise interaction for each pair of sub-units from the data base.

In block 112, routine 100 sorts via the computer processor the pairwise interactions in an ascending order. In an alternative the pairwise interaction may be sorted in a descending order.

In mathematic terms this generates a vector of length N. N represents the number of sampling steps.

In other words, the digital representation is generated by repeatedly sampling two sub-units from the subunits describing the polymer blend N times and retrieving the pairwise interaction for each sampled pair of sub-units from a data base.

In block 114, routine 100 provides the digital representation.

In block 116, routine 100 provides to the processor via the communication interface a data driven model parametrized on a digital representation of historical polymer materials, historical storage times and historical phase stability parameters, the historical storage times are optional. In this example, the digital representations of historical polymer blends is based on a parameter of pairwise interaction of sub-units of the ingredients of the historic polymer material, in this example the polymers in the polymer blend, Block 116 further comprises the step of sampling two sub-units from the sub-units contained in the historic polymer blend and determining the pairwise interaction between the sampled sub-units.

In this example the sub-units contained in the historical polymer blend relate to the relative occurrence of sub-units in the historical polymer blend, which is based on the number of the specific sub-unit in the polymer and the concentration of that polymer in the historical polymer blend. Ideally the number of samplings N historical in the model match the number of samplings N of the digital representation. The pairwise interactions in the model may be sorted in and ascending order. In another example the pairwise interactions may be sorted in a descending order.

In block 118, routine 100 determines with the computer processor a phase stability parameter for the digital representation of the polymer blend based on the provided data driven model, the digital representation of the polymer blend and the target storage time.

In block 120, routine 100 provides via the communication interface the determined phase stability parameter.

In this example the phase stability parameter relates to the probability that the blend is not phase separated after the target storage time. In another example, the probability that the blend is not phase separated after the target storage time may be classified in “stable” and “non-stable” in that case the classifier would be provided as phase stability parameter.

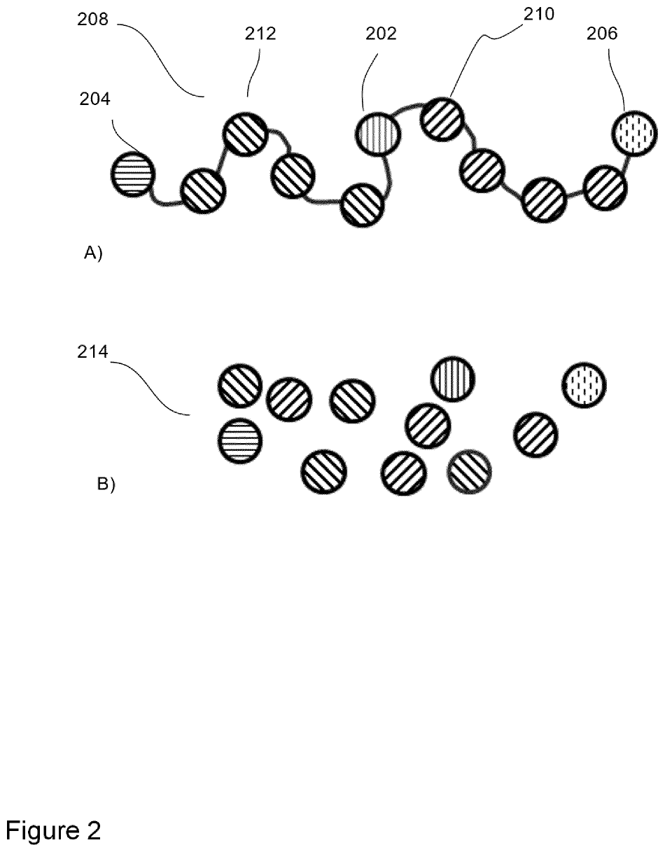

In FIG. 2 an exemplary polymer is depicted. The polymer consists of a cross link 202 and 4 distinct kinds of sub-units; a headgroup 206, a tail group and an a-type sub-unit and a b-type sub-unit. Consequently, per there is only one head group 204 and one tail group 206, the length of the polymer 208 is mainly defined by the number of a-type 210 and b-type 212 sub-units. Similar sub-units are shown with the same hashed pattern.

FIG. 2 b illustrates how the sub-units may be treated for the purpose of the invention. Each sub-unit is considered a particle also named beads. sub-units of the same kind are not distinguishable.

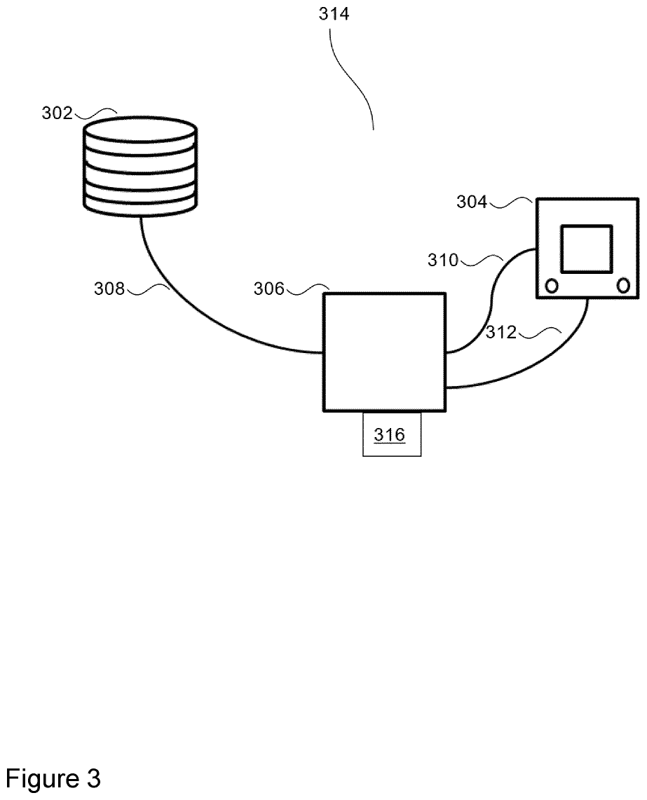

FIG. 3 shows an example of a computing apparatus 300 comprising:
a computer processor 306; a communication interface 308,310, 312 a memory 316 storing instructions that, when executed by the processor, configure the apparatus to perform the steps of provide to a computer processor via a communication interface a digital representation of the polymer material and a target storage time; provide to the processor via the communication interface a data driven model parametrized on a digital representations of historical polymer blends, historical storage times and historical phase stability parameters; determine with the computer processor a phase stability parameter for the digital representation of the polymer blend based on the provided data driven model, the digital representation of the polymer blend and the target storage time provide via the computer processor the determined phase stability parameter.

In this example the computer apparatus further comprises in input/output device 304. In this example the data driven model is stored in a data base 302. The data base 302 is connected to the computer processor via the communication interface 308. In this example input/output device 304 is used to provide a digital representation of a polymer blend to the computer processor 306 via communication interface 310 and a target storage time. In this example the digital representation is provided in the form of the brand name of the polymers in the polymer blend and the concentration of each polymer in the polymer blend. The polymer blend in this example consists of only two polymers. The computer processor 306 then retrieves from the data base 302 the sub-units for each of the polymers. The computer processor 306 derives sub-units from the formulation of the polymer material reflecting the ingredients of the polymer material in sub-units and reflecting the concentration of the ingredients by the concentration of the respective sub-units.

With the computer processor two sub-units from the sub-units contained in the polymer blend are sampled and the pairwise interaction between the sampled sub units is determined.

In this example the pairwise interaction for each sampled two sub-units is retrieved from the data base 302. The sampling is repeated by the computer processor N times. In this example N equals to 1000. The pairwise interactions are then sorted by the processing device in an ascending order.

The data driven model is provided to the computer processor 306 via the communication interface 308. With the computer processor 306 a phase stability parameter is determined.

In this example the phase stability parameter is provided to the input/output device 304 to the input/output device 304 via communication interface 312. In another example the phase stability parameter may be provided to the data base 302 via communication interface 308.

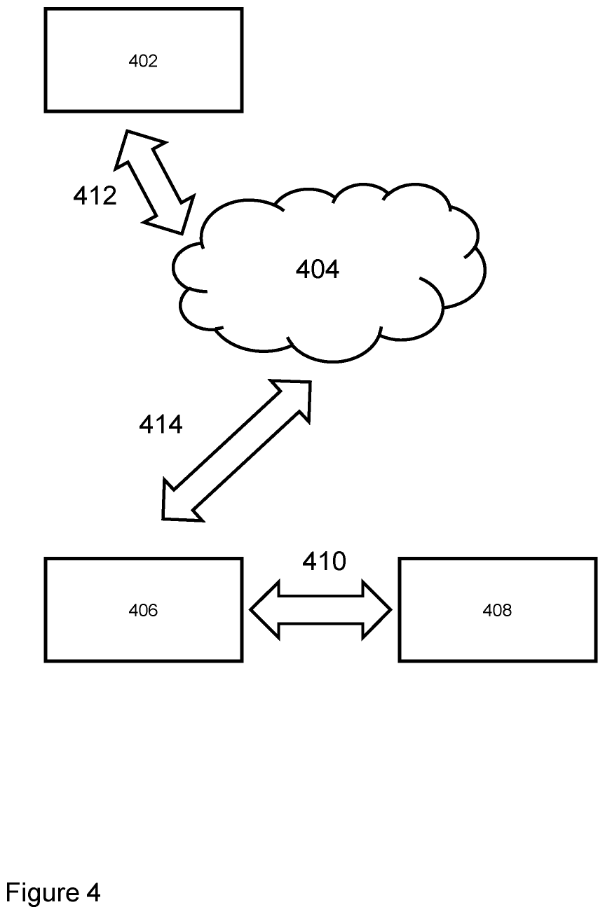

Turning to FIG. 4, there is shown an internet-based system for determing a phase stability factor. The system 400 comprises a server 402 which can be accessed via a network 404, such as the Internet, by one or more clients 406.1 to 406.n. Preferably, the server may be an HTTP server and is accessed via conventional Internet web-based technology. The clients 406 are computer terminals accessible by a user and may be customized devices, such as data entry kiosks, or general-purpose devices, such as a personal computer. A printer 408 can be connected to a client terminal 406. The internet-based system is in particular useful, if a service is provided to customers or in a larger company setup. A client may be used to provide the digital representation of the polymer material to the computer processor of the server.

In an alternative, an internet-based system similar to the system of FIG. 4 may be used for determining a recommended shelf life.

1. A computer implemented method of predicting a phase stability parameter for a polymer material comprising:
providing to a computer processor via a communication interface a digital representation of the polymer material associated with a synthesis specification;
providing to the computer processor via the communication interface a data driven model parametrized on
a digital representation of historical polymer material, and
historical phase stability parameters; and a pairwise interaction parameter associated with the digital representation of the polymer material,
determining with the computer processor a phase stability parameter for the polymer material based on
the provided data driven model, and
the digital representation of the polymer material
providing via the communication interface the determined phase stability parameter.

2. The computer implemented method of claim 1, wherein providing the digital representation of the polymer material comprises providing a formulation of the polymer material.

3. The computer implemented method of claim 2, further comprising deriving sub-units from the formulation of the polymer material.

4. The computer implemented method of claim 1, wherein providing the digital representation comprises reflecting the ingredients of the polymer material in sub-units and reflecting the concentration of the ingredients by the concentration of the respective sub-units.

5. The computer implemented method of claim 4, further comprising providing a parameter for pairwise interaction between sub-units.

6. The computer implemented method of claim 5, wherein the digital representation is based on the parameter of pairwise interactions of sub-units of the ingredients in the polymer material.

7. The computer implemented method of claim 6, further comprising sampling with the computer processor two sub-units from the sub-units contained in the polymer material and determining the pairwise interaction between the sampled sub-units.

8. The computer implemented method of claim 7, comprising repeating the step of sampling and determining N times, wherein N is >=1.

9. The computer implemented method of claim 7, wherein determining the pairwise interaction between the sampled sub-units comprises retrieving the pairwise interaction parameters for a pair of sub-units from a database.

10. The computer implemented method of claim 1, further comprising
providing a targetstorage time, and
and wherein the phase stability parameter is storage time.

11. The computer implemented method of claim 1, wherein the polymer material comprises isocyanates.

12. The computer implemented method of claim 1, wherein the polymer material comprises at least one of polyols polyurethane, polyolefin and its copolymers, polyacrylate, polyester, polycarbonate, polyether and polyacrylics.

13. A computing apparatus comprising:
a computer processor; and
a memory storing instructions that, when executed by the processor, configure the apparatus to perform the steps of the computer implemented method of claim 1.

14. A non-transitory computer-readable storage medium, the computer-readable storage medium including instructions that when executed by a computer, cause the computer to perform the method of claim 1.

15. A system comprising a polymer material and a storage time, wherein the storage time was validated depending on the phase stability parameter determined according the method of claim 1.

16. (canceled)

17. A system comprising:
a polymer material, and
a phase stability parameter, wherein the phase stability parameter is determined according to the method of claim 1.

18. A system comprising:
at least one polymer and/or prepolymer and
a formulation comprising the at least one polymer and/or prepolymer, and
a phase stability parameter, wherein the phase stability parameter is determined according to the method of claim 1.

19. A computer implemented method for determining a formulation of a polymer material, comprising:
providing to a computer processor via a communication interface an initial digital representation of a polymer material,
providing to a computer processor via a communication interface a target phase stability parameter;
providing to the processor via the communication interface a data driven model parametrized on a digital representation of historical liquids containing polymers, historical phase stability parameters; and a pairwise interaction parameter associated with the digital representation of the polymer material,
determining with the computer processor a phase stability parameter for the polymer material based on the provided data driven model, and the digital representation of the polymer material;
optimizing the storage time, by modifying the ratio of the ingredients in the initial digital representation of the polymer material until the desired shelf life is reached;
providing a target formulation comprising the ratio of the ingredients determined in the step of optimizing.

20. A formulation of a polymer material, wherein in the formulation was derived according the method of claim 18.

21. A client device for generating a request to initiate the determination of a phase stability parameter of a polymer material at a server device, wherein the client device is configured to provide a digital representation of the polymer material and the target storage time to a server device.
